(19) United States
(12) Patent Application Publication
(10) Pub. No.: US 2011/0230972 A1
(43) Pub. Date: Sep. 22, 2011

(54) ELBOW RESURFACING PROSTHESIS

(75) Inventors: Nicholas J. Katrana, Fort Wayne, IN (US); Thomas M. Vanasse, Warsaw, IN (US); Adam Finley, Leesburg, IN (US); William J. Hamman, Winona Lake, IN (US); Robert Metzger, Wakarusa, IN (US); Bryce A. Isch, Bluffton, IN (US)

(73) Assignee: BIOMET MANUFACTURING CORP., Warsaw, IN (US)

(21) Appl. No.: 12/884,696

(22) Filed: Sep. 17, 2010

Related U.S. Application Data
(60) Provisional application No. 61/243,913, filed on Sep. 18, 2009.

(51) Int. Cl. A61F 2/38

(52) U.S. Cl. 623/20.11

(57) ABSTRACT

A method and apparatus for replacing a selected portion of the anatomy is described. In particular, a prosthesis can be provided to replace a portion of an articulating joint, such as an elbow. The apparatus can be modular for various reasons and each of the modular portions can include a different dimension to achieve a selected result.

### CROSS-REFERENCE TO RELATED APPLICATIONS

This application claims the benefit of U.S. Provisional Application No. 61/243,913, filed on Sep. 18, 2009. The entire disclosure of the above application is incorporated herein by reference.

### FIELD

The present teachings relate generally to prosthetic devices used in arthroplasty and more particularly to a modular elbow prosthesis.

### BACKGROUND

Elbow prostheses are known which comprise simple hinge arrangements, one component of which is attached to the end of the humerus and the other component of which is attached to the end of the ulna. The humeral component includes a shaft, that is cemented into a prepared cavity in the end of the humerus, and the ulnar component includes a shaft, that is cemented to the end of the ulna. The components of the prosthesis are connected together by means of a hinge pin so that the prosthesis allows a single degree of freedom of movement of the ulna relative to the humerus.

Often the use of these prostheses requires a removal of significant amounts of bone. While reducing bone removal may be contemplated, the specific physiology of the elbow joint significantly increases complications related to bone removal and slows recovery time.

### SUMMARY

To overcome these and other deficiencies of the prior art, an elbow prosthesis constructed in accordance with one example of the present teachings which includes a capitellum implant having an articulating head is provided. The articulating head can have a first articulating surface positioned generally between an anterior side and a posterior side of the humerus. A faceted medial bearing surface is provided which interfaces with a prepared humeral surface.

In another embodiment, the present teaching provides a method for resurfacing a capitellum. The method includes preparing the capitellum and implanting an implant at the prepared surface. The implant has an exterior articulating surface, an interior surface opposite the exterior surface, and a fixation mechanism. The interior surface of the implant defines a pair of intersected planar surfaces. Optionally, the implant can have a stem configured to be implanted into an intermedullary canal.

Further areas of applicability of the present invention will become apparent from the detailed description provided hereinafter. It should be understood that the detailed description and specific examples, while indicating a preferred embodiment, are intended for purposes of illustration only and are not intended to limit the scope of the invention.

### DRAWINGS

The drawings described herein are for illustrative purposes only of selected embodiments and not all possible implementations, and are not intended to limit the scope of the present disclosure.

FIGS. 1-4 represent a capitellum resurfacing head according to the present teachings;

FIG. 5 represents a cutting guide which is used to prepare the capitellum for use with the resurfacing implant of FIGS. 1-4;

FIGS. 6A-6D represent perspective views of various prepared humerus;

FIG. 7 shows a cross-sectional view of the implantation of the resurfacing head shown in FIGS. 1-4;

FIG. 8 represents a side view of an alternate humeral resurfacing bearing;

FIG. 9 represents an end view of the bearing shown in FIG. 8;

FIG. 10 represents a side view of an alternate resurfacing head coupling mechanism;

FIGS. 11A and 11B represent side cross-sectional views of the coupling of the resurfacing prosthetic of FIG. 9 to a humerus;

FIGS. 12A and 12B represent perspective views of an alternate resurfacing prosthetic;

FIGS. 13A-13D represent perspective, top, and side views of an alternate resurfacing prosthetic;

FIG. 14 represents a cross-sectional view of the resurfacing bearing of FIG. 9;

FIGS. 15A-15H represent cross-sectional views of the resurfacing bearing of FIG. 9;

FIGS. 16A-16E represent perspective end, side and sectional views of the resurfacing bearing of FIG. 9 with an alternate coupling mechanism;

FIGS. 17-20 represent the use of a first cutting guide according to the present teachings;

FIG. 21 represents the use of a second cutting guide according to the present teachings;

FIG. 22 represents a prepared humerus;

FIGS. 23 and 24 represent the implementation of the prosthetics according to the present teachings;

FIGS. 25-28 represent an alternate method of preparing a humerus;

FIG. 29 represents an alternate prosthetic cross-section; and

FIGS. 30-32 represent the use of a cutting guide according to the present teachings.

Additional advantages and features of the present teachings will become apparent from the subsequent description and the appended claims, taken in conjunction with the accompanying drawings.

### DETAILED DESCRIPTION OF VARIOUS EMBODIMENTS

The following description is merely exemplary in nature and is not intended to limit the present disclosure, application, or uses. It should be understood that throughout the drawings, corresponding reference numerals indicate like or corresponding parts and features.

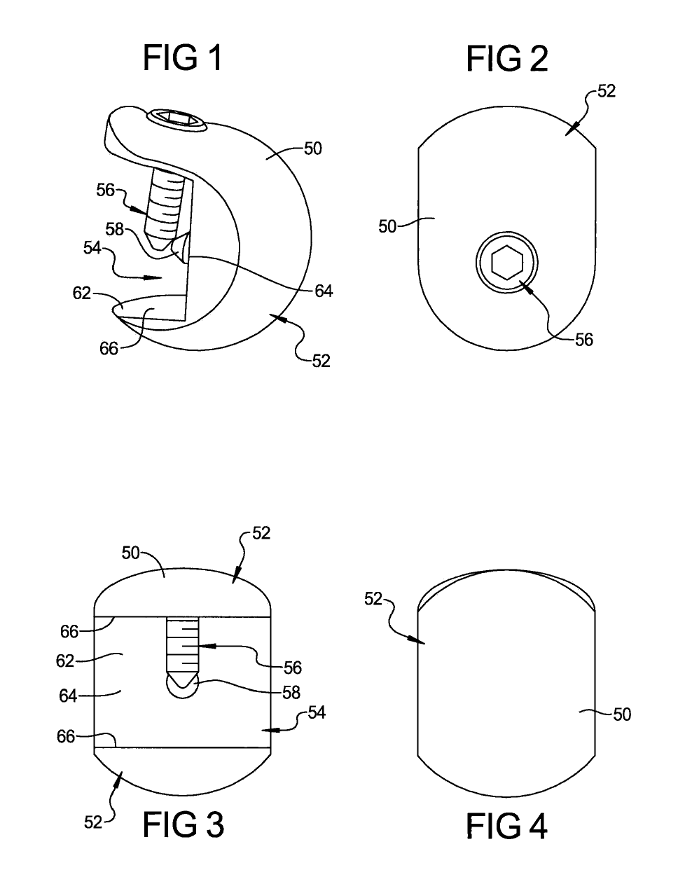

Referring to FIGS. 1-4, a resurfacing capitellum implant 50 according to the present teachings is provided. The capitellum implant 50 has a first articulating surface 52 and second coupling side 54 adapted to be coupled to a prepared surface of a capitellum. The resurfacing capitellum implant 40 has a fixation mechanism 56 which is used to facilitate the fixation of the resurfacing capitellum implant 50 to the prepared capitellum surface.

The fixation mechanism 56 can be a centrally disposed fixation peg 58 and/or at least one bone fixation screw 56. As shown in FIG. 3, the fixation screw 56 can be configured to engage the prepared humeral surface at a predetermined angle with respect to a fixation peg 58. Optionally, the screw 56 can be positioned generally perpendicular to the centrally disposed fixation peg 58 on either an anterior or posterior surface of the humerus. Additionally, the fixation peg 58 can include porous plasma spray with or without Hydroxyapatite, stem or pegs (porous metal that is fixed or modular) and/or a locking screw on the anterior or posterior side of the capitellum. As described below, the posterior side of the prosthetic can be extended to allow for the use of a bone fixation screw. Fixation members can be porous coated to encourage boney in-growth.

The second coupling side 54 of the implant 50 can be formed of more than one intersecting coupling surfaces 62. In this regard, the intersecting surfaces 62 can be a first medial surface 64 with a pair of generally perpendicular surfaces 66. It is envisioned the intersecting perpendicular surfaces 66 can intersect the medial surface 64 at an angle from about 20° to 5° and, more particularly, at about 10° from normal, to facilitate the coupling of the prosthetic to a prepared humerus.

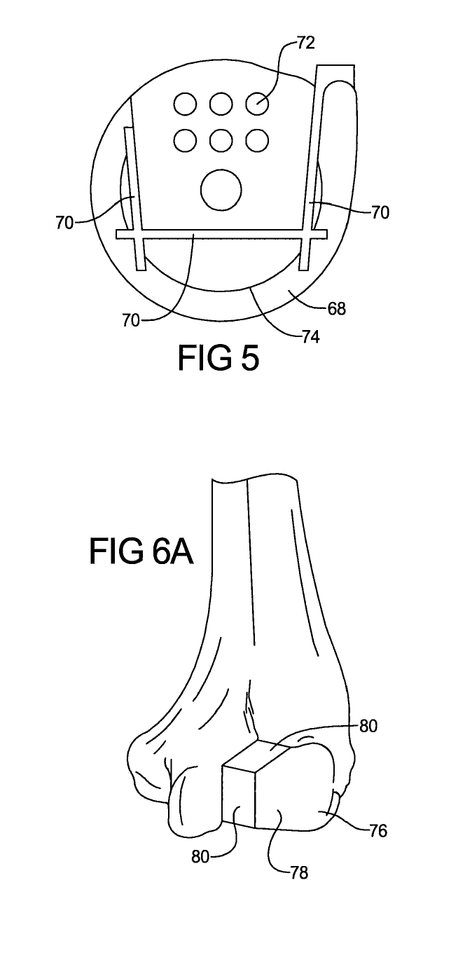

FIG. 5 represents a cutting guide 68 for use to prepare a capitellum. The cutting guide 68 has a plurality of cutting slots 70 which correspond to the coupling surfaces 64 and 66 of the second coupling sides 54. Additionally shown are a plurality of holes 72 for coupling the cutting guide 68 to an unprepared capitellum surface using pins. The cutting guide 68 defines an interior spherical or cylindrical surface 74 which is configured to bear against the unprepared capitellum. It is envisioned the cuts can be made from lateral to medial through the cutting slots 70.

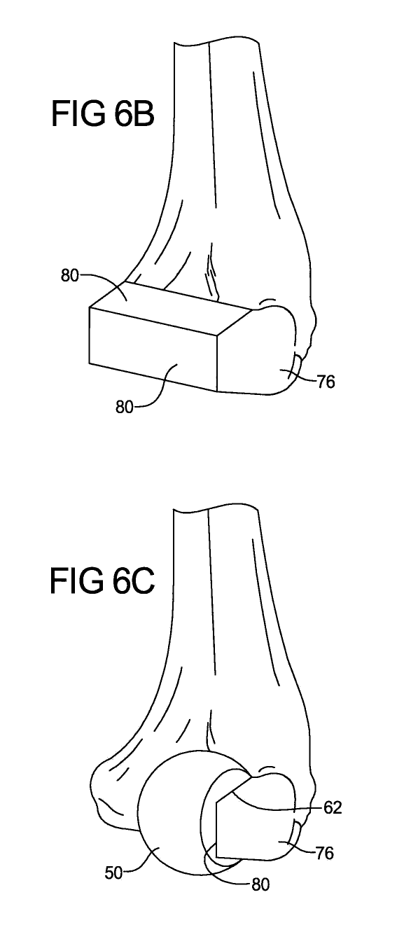

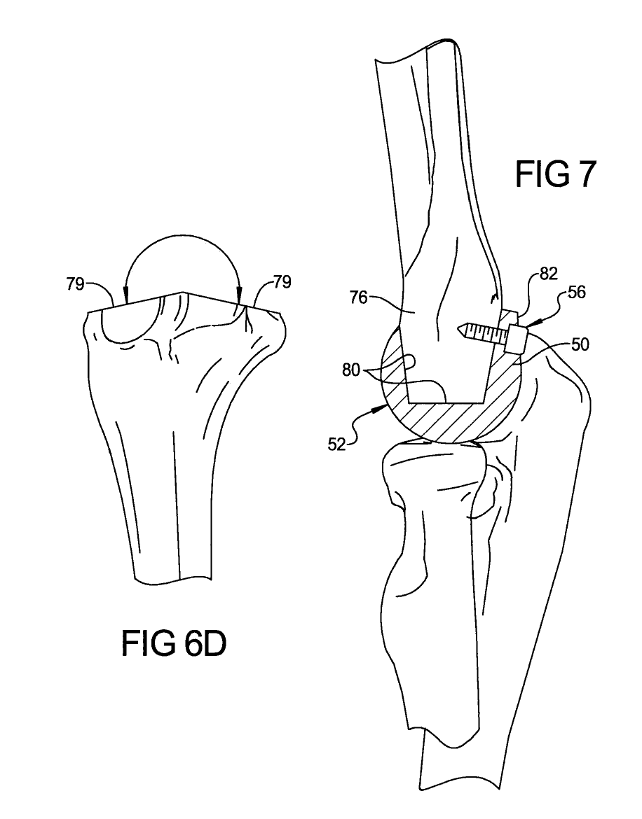

FIGS. 6A and 6B represent perspective images of a prepared humerus 76. In this regard, optionally the surface of a resected capitellum 78 has three intersecting bearing surfaces 80 that can be formed using the cutting guide shown in FIG. 5. Alternatively, as described further below and shown in FIG. 6B, the resection can also be performed over other elbow articulating surfaces such as the trochlea or internal condoyle.

Optionally, the medial/lateral cut of the humeral head can be plain or angled. As shown in FIG. 6D, the humerus can be resected using a pair of angled medial to lateral cuts 79. The angled cuts reduces loading on the distal radius. FIGS. 6C and 7 represent perspective and cross-section views of the capitellum implant 50 coupled to the three intersecting bearing surfaces 80 of the prepared capitellum. The internal surfaces 62 of the coupling side 54 are engaged with the prepared surfaces 80. Further shown is the fixing of the implant 50 using a bone fixation screw 56.

As best seen in FIG. 7, the resurfacing implant 50 can have an extended coupling surface 82 which defines a bone screw 56 accepting aperture. The resurfacing implant can have an articulating surface 52 which is configured to interface with an articulating surface of a natural or prosthetic radial articulating surface. Optionally, the extended coupling surface can be polished to allow it to interface with an articulating bearing surface (not shown). The articulating surface 52 is configured to bear against a natural or prosthetic radial articulating surface.

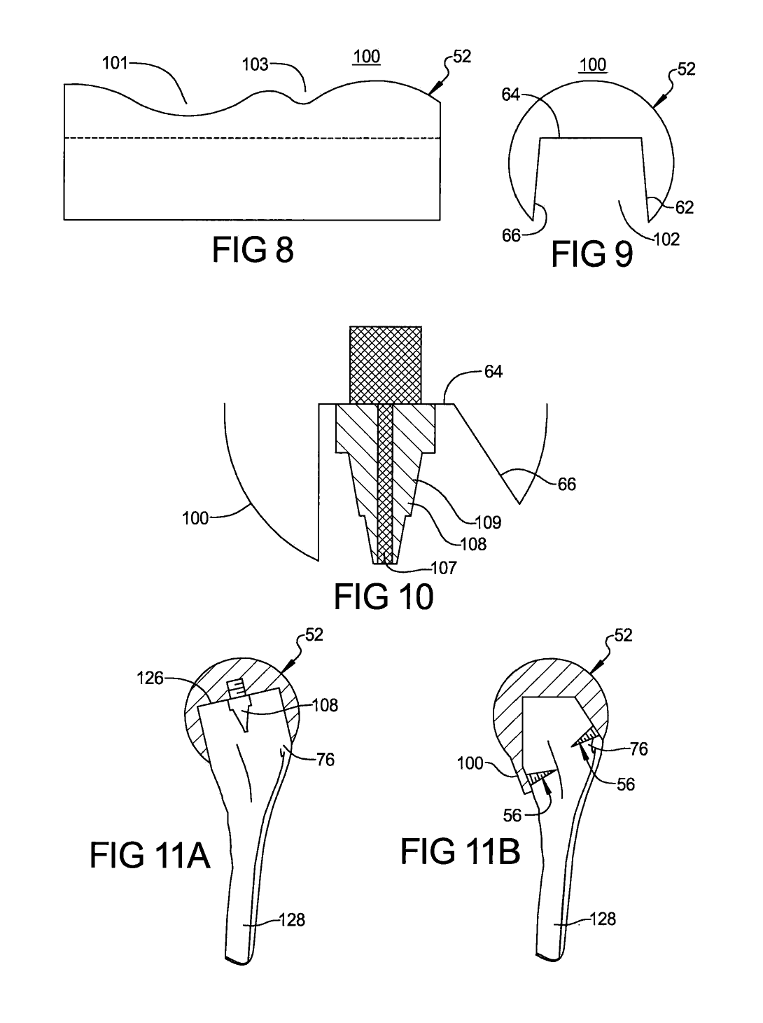

FIGS. 8 and 9 represent side and end views of an alternate resurfacing implant 100 according to the present teachings. Shown is an implant configured to replace the surface of the capitellum and trochlea. The implant 100 defines a coupling groove 102 having interface surfaces 62, 64, and 66. As described below, humeral surfaces configured to mate with the interface surfaces can be formed by using cutting guides to define angles as described above. The trochlea portion 101 is configured to articulate with a natural or prosthetic ulna, while the capitellum region 103 is configured to articulate with a natural or prosthetic radius.

As shown in FIGS. 10-11B, the resurfacing implant 100 can have associated fixation pegs 108. Alternately, fixation screws 56 can be used to couple the implant 100 to the prepared capitellum and trochlea. The fixation peg 108 can be formed of a single or multiple interior titanium posts 107 with a powder metal exterior 109. The peg 108 can be threadably coupled to the implant 100. It is envisioned the peg 108 can be encapsulated within the groove 102 and, as such, not enter the humeral intermedullary canal upon implantation.

FIGS. 11A and 11B represent the coupling of the implant 100 to a prepared humeral surface. The implant 100 can have a pair of modular or integral central pegs 108 which are fixed into bores defined in the prepared interface surfaces. As shown, the resurfacing implant 100 can be coupled to the prepared surfaces using bone engaging screws 56. Optionally, the implant can have extended fixation surfaces 110 defining bone fastener engaging apertures 112. The bone engaging screws can be implanted through the depending fixation flange 110 or through a hole defined in the articulating surface.

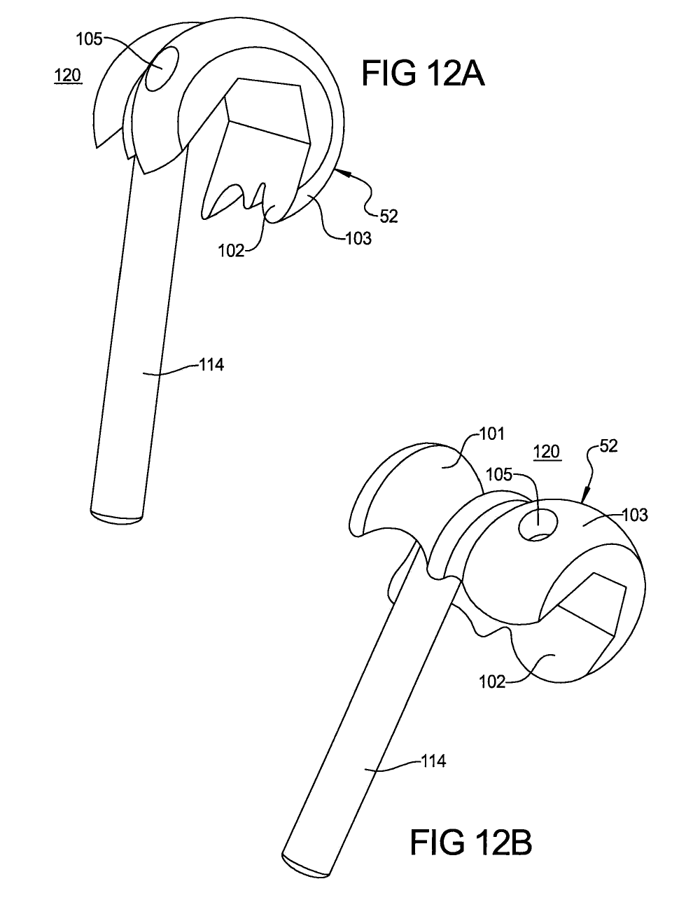

As shown in FIGS. 12A and 12B, an alternate prosthetic 120 can have a depending coupling stem 114. The prosthetic 120 has bearing and articulating surfaces as described above. The stem 114 is configured to be positioned within a medullary canal defined in the humerus. Optionally, the stem 114 can be offset with respect to the rotation center of the prosthetic 120. In this regard, stem 114 can project off of one of the coupling surfaces, the location of which is set to maintain proper articulation of the elbow joint. Also shown is a bone screw accepting aperture 105 defined in an articulating surface 52.

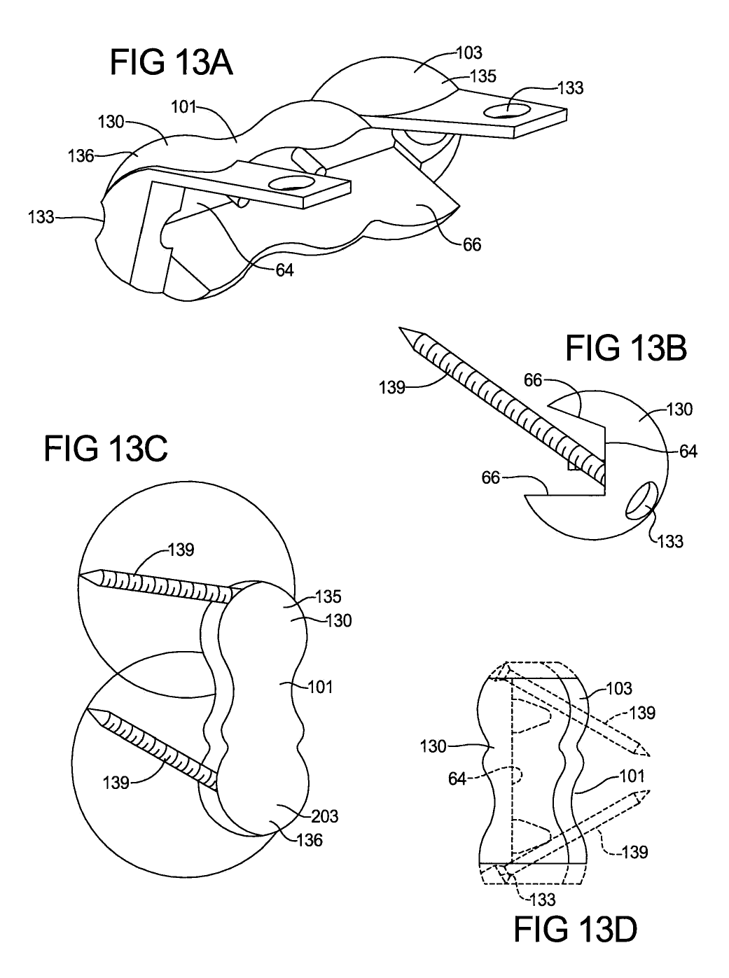

FIGS. 13A-13D represent an alternate resurfacing implant 130 according to the present teachings. The implant 130 has a trochlea portion 101 and a capitellum region 103 configured to articulate with a natural or prosthetic radius. The coupling surface 64 and 66 can have a coupling mechanism as described throughout this application. Optionally disposed on the implant 130 is a pair of exterior flanges 132. The flanges 132 define a bone screw accepting aperture 133.

Medial and lateral sides 135 and 136 of the implant 130 define side support members 137 and 138 which can define bone screw accepting apertures 133. As seen in FIGS. 13B and 13C, the bone screw accepting apertures 133 can be configured to allow the bone engaging screw 139 to enter the humerus to enter the bone at varying number of angles. The apertures 133 are positioned medially and laterally so as to not interfere with the ulna and radius (natural or prosthetic.)

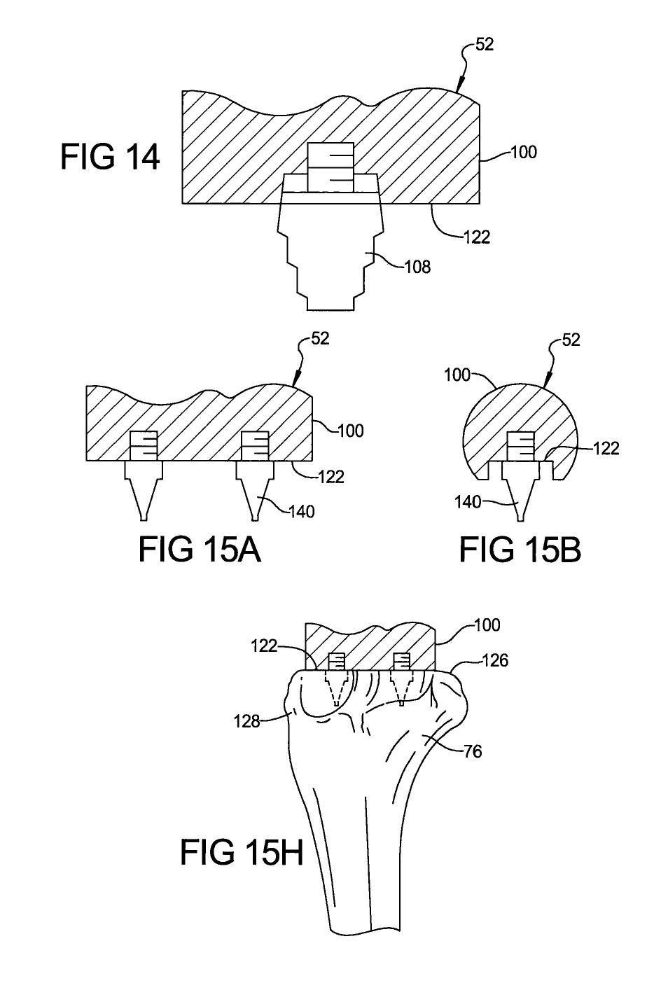

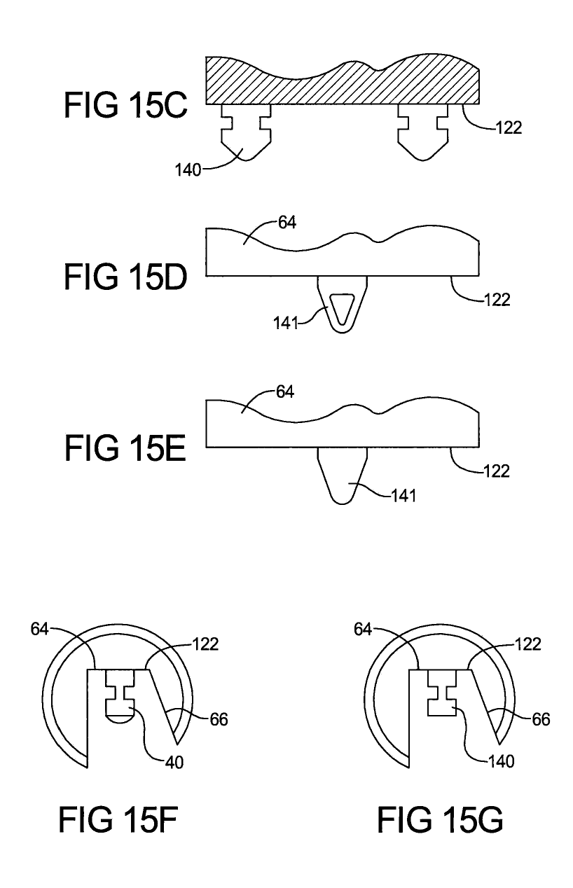

FIGS. 14-15H show cross-sections of the humeral prosthetic shown in FIGS. 8 and 9 with varying coupling mechanisms. In this regard, the prosthetic 100 can have a flat interface surface 122. The interface surface 122 can have a pair of modular or integral fixation pegs 108 configured to couple the implant to a resected surface 126 of the humerus 128. Wedge shaped keels can include undercuts in both A-P view or M-L view to allow for cement adhesion and locking geometry for bone cement.

As shown in FIG. 14, a peg accepting bore can be countersunk and define a wedge which corresponds to a mating locking wedge of the peg 108. The articulating surface can be formed of cobalt or PEEK/CFR/PEEK/Polycarbon. Additionally, the articulating surface can be injected molded PEEK/CFR-PEEK over a metallic substrate which mates with bone and posts threaded therein.

FIGS. 15A and 15H represent cross-sectional views of the resurfacing head prosthetic. The resurfacing head prosthetic has a generally cylindrical body defining a through axis. Optionally disposed on the medial coupling surface 122 is the coupling mechanism which can intersect the through axis of the cylindrical body. The coupling mechanism can be a pair of coupling pegs 140. The pegs 140 which can be tapered, stepped, or cylindrical are configured to be implanted into a pair of holes defined in a resected surface of the humerus. As shown in FIGS. 15D and 15E, the coupling mechanism can be a single or pair of keels 141. The keels 141 are thin and wedge shaped, that optionally can define a window for porous metal. Optionally, the keels 141 can be coated with titanium plasma spray.

FIG. 15F represents a cross-sectional view of the coupling of the prosthetic of FIGS. 15A and 15B into the resected humerus 76. As shown, after resecting of the outer surface of the humerus, a pair of holes 144 can be defined therein. Disposed within the pair of holes can be the coupling pegs 140.

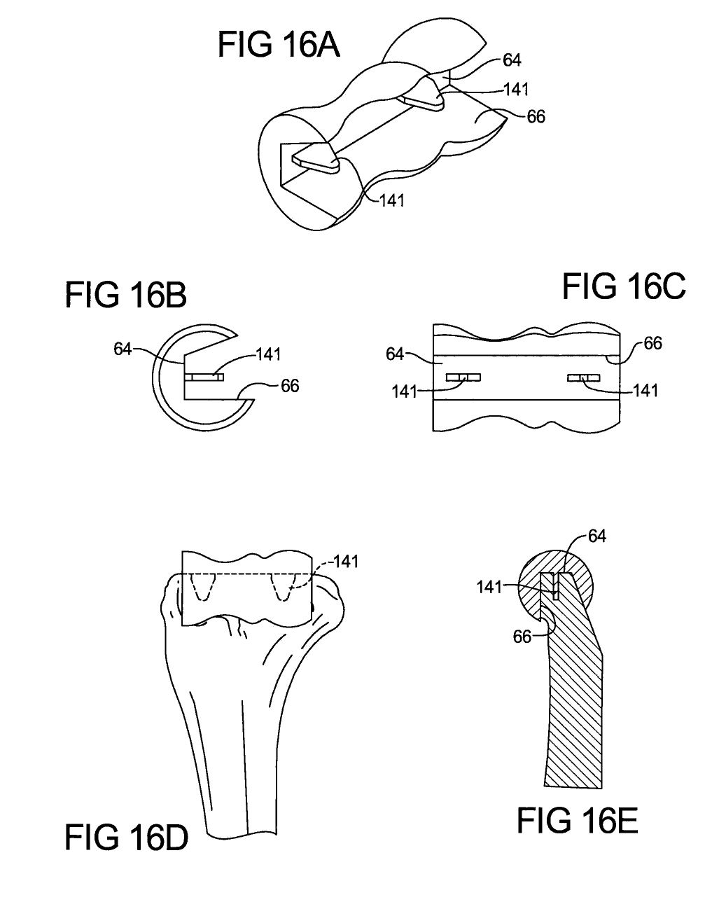

FIGS. 16A-16E represent a perspective, side, end, and cross-sectional views of the prosthetic shown in FIGS. 15A-15H. Included are a pair of keels 141 which are used to couple the prosthetic to the resected humerus. If two keels 141 are used, it is envisioned to place them in the medial and lateral columns of the distal humerus where there is sufficient bone stock. The distal humerus can be prepared utilizing a template guide to locate the position of the keel. A sharp punch (or rasp with teeth) can be used to create a cavity to accept the keels 141. Additionally, the cavity can be formed by a rotatable tool such as a drill.

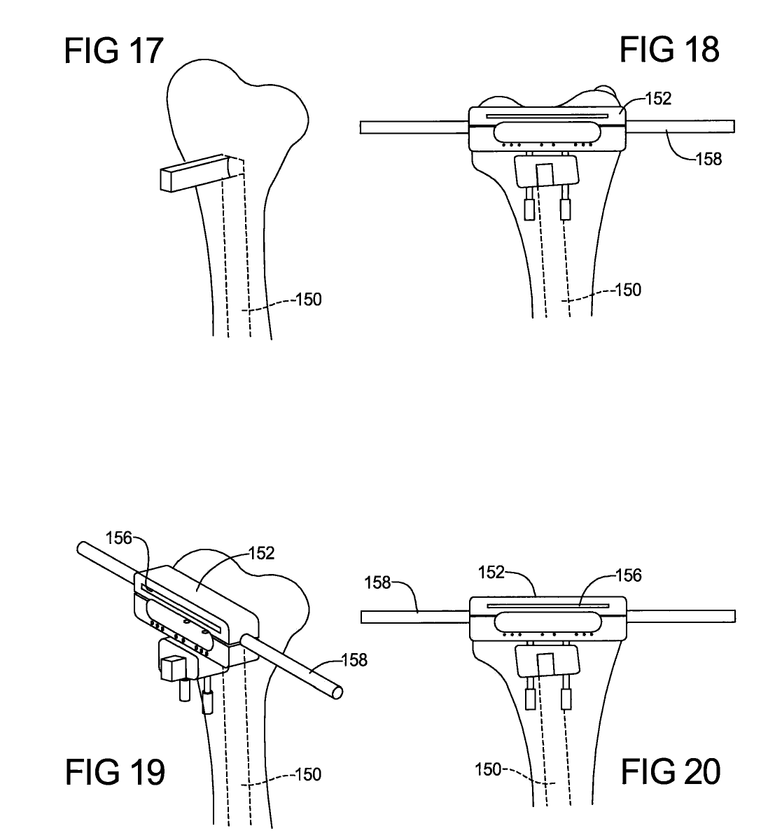

FIGS. 17-32 represent the preparation of the humerus with associated cutting fixtures. As shown in FIG. 17, an alignment pin 150 can be positioned through a hole defined in a wall of the humerus and into the medullary canal. As shown in FIG. 18, an anteriorly positioned first cutting guide 152 is coupled to the alignment pin 150. The alignment pin 150 can be accepted by an aperture 154 defined within a cutting guide 152.

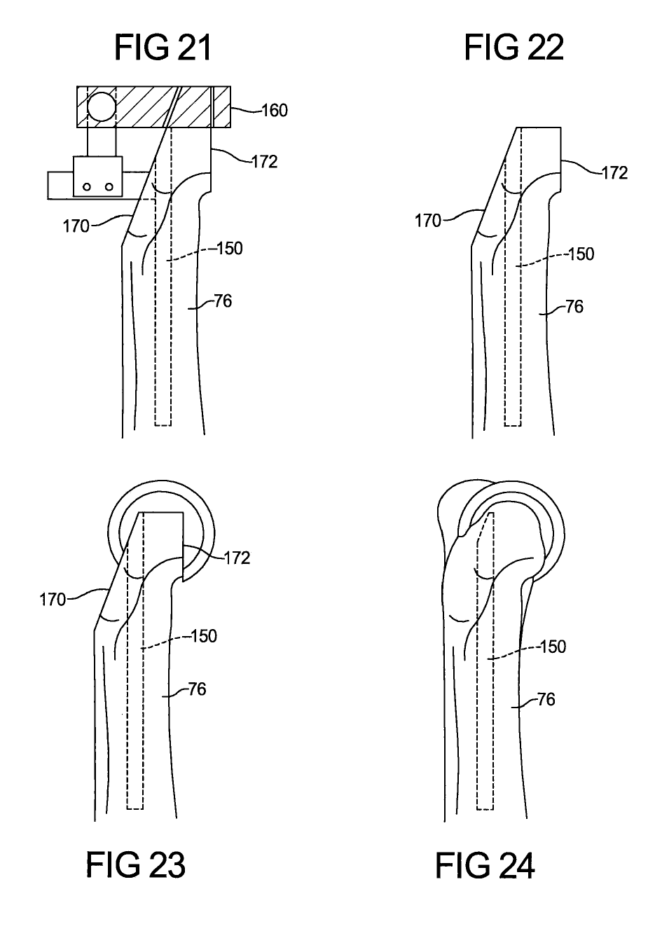

As seen in FIGS. 19 and 20, the cutting guide 152 can define a slot 156 and can support an alignment bar 158. The slot 156 is used to form a humeral distal flat cut. The bar 158 is optionally used to align the rotating cutting tools used to form flat surfaces (see FIGS. 21 and 22) or curved bearing surfaces 25-28.

FIGS. 21 and 22 represent a second cutting guide 160 configured to allow anterior and posterior cuts on the humerus and associated resections. The cutting guide 160 is coupled to the alignment pin 150 positioned within the medullary canal. After adjustment of the first portion to the resected distal end, resections 170 of the anterior and posterior sides can be made. These resections correspond to surfaces within the resurfacing prosthetic. As shown in FIGS. 22-24, once the resections are made, the implants described herein can be coupled to the resected surfaces either with or without fixative cement.

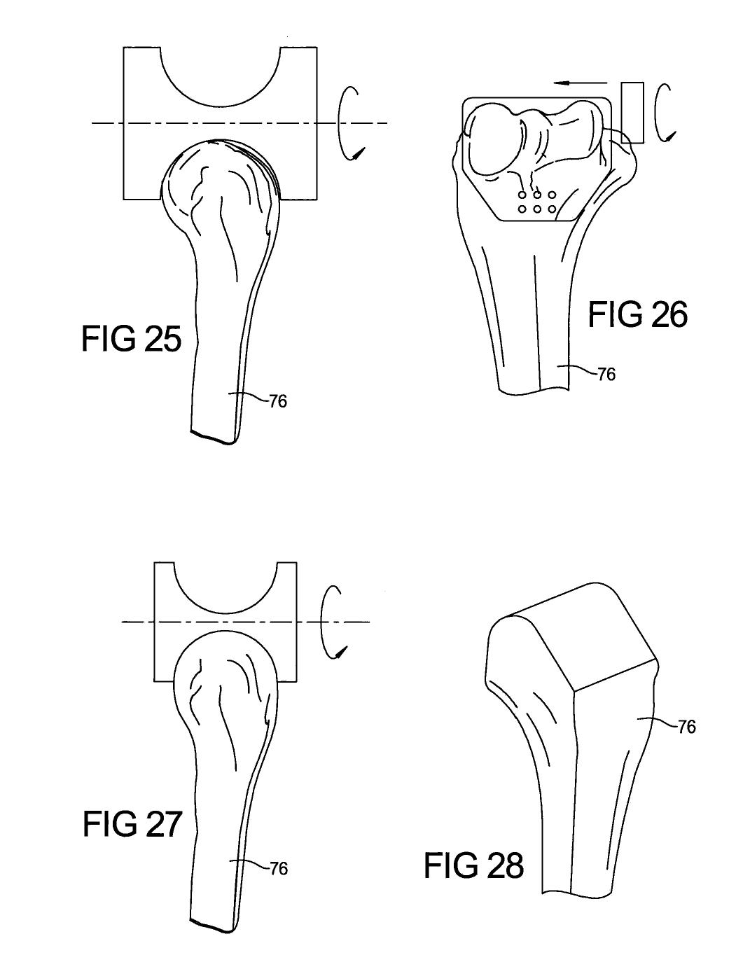

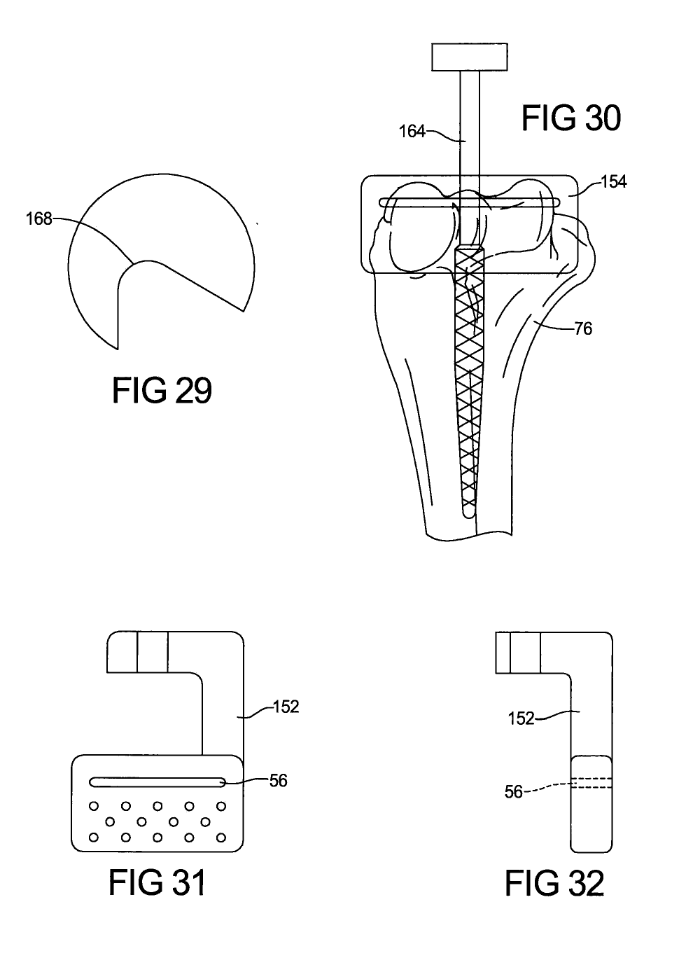

As shown in FIGS. 25-29, rotating cutting members guided by the alignment bar 158 can be used to form a curved distal bearing surface 180. On anterior and posterior sides of the humerus, generally flat bearing surfaces can be resected therein. Corresponding interior bearing surfaces (see FIG. 29) can be formed into either the capitellum or capitellum/trochlea implants. These intersecting surfaces can be coupled through a bearing interface curve 168.

As seen in FIGS. 30-32, the cutting guides 152 and 162 can be configured to rest on the handle of a rasp 164 as opposed to the alignment pin 150. It is envisioned this combination of a cutting tool and alignment guide reduces operation procedure time. Additionally, it is envisioned the handle of the rasp 164 can have an associated perpendicular member which allows the use of cutting guides 152 and 162.

While the description in the specification and illustrated in the drawings are directed to various embodiments, it will be understood that various changes may be made and equivalents may be substituted for elements thereof without departing from the scope of the teachings and the appended claims. In addition, many modifications may be made to adapt a particular situation or material to the teachings without departing from the scope thereof. Therefore, it is intended that the teachings and claims are not be limited to any particular embodiment illustrated in the drawings and described in the specification, but that the teachings and claims can include any embodiments falling within the foregoing description and the appended claims.

What is claimed is:

1. An elbow prosthesis comprising:
a capitellum implant including an articulating head, having a first articulating surface and a medial coupling portion, said coupling portion having a plurality of intersecting bearing surfaces.

2. The elbow prosthesis of claim 1, wherein the articulating head further defines a bore configured to accept a bone engaging screw.

3. The elbow prosthesis of claim 1, further comprising a stem configured to be implanted in an intermedullary canal of a humerus.

4. The elbow prosthesis of claim 1, wherein the capitellum implant has a first interlocking geometry formed at the medial coupling portion.

5. The elbow prosthesis of claim 1, wherein the capitellum implant further comprises an extension portion that extends from the articulating surface, the extension portion defining a bone screw accepting aperture.

6. A prosthesis to replace an end portion of a bone, comprising:
a generally cylindrical bearing member having an exterior articulating surface and a medial coupling portion operable to replace a selected portion of a distal humerus, said coupling portion defining a plurality of intersecting planar surfaces, the cylindrical bearing member including a first articulation surface and a second articulation surface, wherein the first articulation surface includes a first substantially smooth surface operable to articulate with at least one of a radius and a radial bone replacement, and the second articulation surface includes a second substantially smooth surface operable to articulate with at least one of an ulna and ulna prosthesis;
a coupling mechanism attached to the medial surface.

7. The elbow prosthesis of claim 6, further comprising a flange portion extending from the articulation member at a location laterally offset from and substantially in the same direction as the stem member.

8. The prosthesis of claim 7, wherein the flange portion defines a bone screw accepting aperture.

9. The prosthesis of claim 7, wherein the flange portion is formed as a an integral piece with the articulation member.

10. The prosthesis of claim 7, wherein the connecting member is a stem configured to be positioned in a humeral medullary canal.

11. The prosthesis of claim 7, wherein the coupling mechanism comprises at least one stem.

12. The prosthesis of claim 11, wherein the least one stem is integrally formed with the generally cylindrical bearing member.

13. The prosthesis of claim 6, wherein the coupling mechanism is a pair of coupling posts.

14. The prosthesis of claim 7, wherein the generally cylindrical bearing member defines a through axis.

15. The prosthesis of claim 14, wherein the coupling member is a pair of coupling posts which intersect the through axis.

16. The prosthesis of claim 14, further comprising a flange portion extending from the articulation member at a location laterally offset from said through axis.

17. A prosthesis to replace an end portion of a bone, comprising:
a generally cylindrical bearing member having an exterior articulating surface and a medial coupling portion operable to replace a selected portion of a distal humerus, the cylindrical bearing member defining an axis and including a first articulation surface having a first substantially smooth surface operable to articulate with at least one of a radius and a radial bone replacement and a second articulation surface having a second substantially smooth surface operable to articulate with at least one of an ulna and ulna prosthesis; and
a flange portion extending from the articulation member at a location laterally offset from said axis.

18. The prosthesis of claim 17, further comprising a coupling mechanism attached to the medial surface.

19. The prosthesis of claim 18, wherein the coupling mechanism comprises a keel.

20. The prosthesis of claim 19, wherein the keel is aligned with the axis.

21. The prosthesis of claim 17, wherein the flange portion comprises a polished surface configured to articulate on one of operable to articulate with at least one of a radius and a radial bone replacement, and the second articulation surface includes a second substantially smooth surface operable to articulate with at least one of an ulna and ulna prosthesis.
